(19) United States
(12) Patent Application Publication
(10) Pub. No.: US 2025/0381322 A1
(43) Pub. Date: Dec. 18, 2025

(54) EXPANDABLE LINERS

(71) Applicant: Zeus Company LLC, ORANGEBURG, SC (US)

(72) Inventors: Robert L. Ballard, Lexington, SC (US); Patrick Cooper, Orangeburg, SC (US); Dana Barringer, Orangeburg, SC (US); Fuller Prickett, Columbia, SC (US); H. Edward Rast, III, Cameron, SC (US)

(21) Appl. No.: 19/237,335

(22) Filed: Jun. 13, 2025

Related U.S. Application Data
(60) Provisional application No. 63/659,870, filed on Jun. 14, 2024.

(51) Int. Cl. A61L 29/08; A61L 31/10; A61M 25/00

(52) U.S. Cl. CPC A61L 29/085; A61L 31/10; A61M 25/0045

(57) ABSTRACT

A common issue when building a catheter with a laser-cut hypotube is bonding the OD (outer diameter) of the PTFE or PE liner to the ID (inner diameter) of an adjacent component, e.g., a laser-cut hypotube An expandable tie layer or “heat grow tie layer” as described herein can be used as a coating for the liner that will expand or foam up to the ID (inner diameter) of an adjacent component, e.g., hypotube and improve adhesion to the hypotube without impacting the flexibility or lubricity of the liner. This and other applications of the expandable layer are described herein.

### CROSS-REFERENCE TO RELATED APPLICATIONS

The present application claims the benefit of U.S. Provisional Patent Application No. 63/659,870, filed Jun. 14, 2024, which is incorporated herein by reference in its entirety.

### FIELD OF THE INVENTION

The present application is directed to coated catheter liners and methods for making and using such coated catheter liners within catheter assemblies.

### BACKGROUND

Polytetrafluoroethylene (PTFE) has been the ideal material for inner liners of catheters due to the chemical resistance, biocompatibility, and low coefficient of friction (COF) of PTFE. PTFE exhibits unique characteristics in this field that other polymers have not been found to exceed. With such a low COF, PTFE has been able to provide an inner diameter (ID) suitable for catheter liners that easily allows various catheter technologies such as stents, balloons, and atherectomy or thrombectomy devices to be pushed through a small diameter catheter lumen. The effect of low COF/increased lubricity of the catheter inner diameter is a reduced deployment force of catheter devices as catheter devices are passed through the lumen of the catheter ID, increasing the likelihood of a smoother procedure and less patient discomfort.

In catheter constructions, the catheter liner is stretched over a mandrel, which is usually stainless steel or PTFE. The liner can be inserted into a hypotube, a small, flexible metal or polymeric tube (materials, construction, and dimensions may vary for different applications) during catheter construction to enhance its performance, particularly in applications requiring high flexibility, pushability, and torque. A hypotube is often used as a reinforcing element, providing strength and stability while allowing for easy navigation through the body's vasculature. Stainless steel or nitinol hypotubes are commonly used in neurovascular applications that are particularly tortuous. The flexibility of the hypotube is controlled by machining kerfs along the length of the tube by methods known in the art such as laser cutting. The surface of a hypotube is sometimes treated to alter its chemistry, making it more compatible with the material that will be used for the polymer jacket. A tie layer, typically made of the same material and durometer as the catheter jacket, can then be applied to the surface of the hypotube inner diameter or over a catheter liner in order to enhance adhesion. A braided or coiled wire reinforcing layer may be constructed on top of the tie layer and can vary in picks, wire dimensions, and materials for different applications. A catheter jacket is then slid over the underlying layers, followed by a heat shrink tube over the catheter jacket. The finalized construction is then laminated together in a process known as reflow and removed from the mandrel, resulting in a fully built catheter.

Adhesion of the liner to a metallic or polymeric hypotube is critical in allowing the finished catheter to retain the flexibility required to navigate the tortuous vasculature. Etching a PTFE liner or using a tie layer are methods for achieving better adhesion between a liner and hypotube. Techniques such as stretching the liner during production can reduce its bondability and thus reduce adhesion and increase the risk of delamination, which can cause device failure and jeopardize patient safety. Commonly, a heated mandrel or a balloon is used to force the liner and tie layer against the inner diameter (ID) of the hypotube, and this process poses challenges such as over-stretching the liner and tight dimensional tolerances or clearance needed to achieve precise fit between the liner, tie layer, and hypotube. Furthermore, the high lubricity needed for insertion of a catheter liner into a hypotube necessitates the frequent use of fluoropolymers such as PTFE in catheter liners. This lubricity requirement in turn limits the materials that can be utilized as catheter liners due to the tight clearance between the liner outer diameter (OD) and hypotube inner diameter (ID) that current bonding methods demand. A more robust solution for easier insertion of a liner into a hypotube that achieves strong adhesion and does not necessitate the use of fluoropolymers is needed.

### SUMMARY

The present disclosure relates to modified tubings, e.g., catheter liners that exhibit unique properties associated with an expandable tie layer coating on at least a portion of an outer surface thereof. The expandable tie layer coating can be expanded to provide modified catheter assemblies. The disclosure includes, without limitation, the following embodiments.

Embodiment 1: A coated tubing comprising: a polymeric tubing, the polymeric tubing having an inner surface and an outer surface and a wall thickness; and an expandable tie layer coating on at least a portion of the outer surface that can expand about 5% or more in outer diameter when subjected to heat.

Embodiment 2: The coated tubing of Embodiment 1, wherein the expandable tie layer can expand about 10% or more in outer diameter when subjected to heat.

Embodiment 3; The coated tubing of Embodiment 1 or 2, wherein the expandable tie layer can expand about 15% or more in outer diameter when subjected to heat.

Embodiment 4: The coated tubing of any of Embodiments 1-3, wherein the expandable tie layer can expand up to about 65% in outer diameter when subjected to heat.

Embodiment 5: The coated tubing of any of Embodiments 1-4, wherein the polymeric tubing comprises polytetrafluoroethylene (PTFE) or polyethylene (PE).

Embodiment 6: The coated tubing of any of Embodiments 1-5, wherein the polymeric tubing comprises polyurethane (PU) or polyether block amide (PEBA).

Embodiment 7: The coated tubing of any of Embodiments 1-6, wherein the expandable tie layer coating is on substantially all of the outer surface.

Embodiment 8: The coated tubing of any of Embodiments 1-7, wherein the expandable tie layer coating comprises a bonding agent and an expansive component.

Embodiment 9: The coated tubing of Embodiment 8, wherein the bonding agent comprises a polymer selected from a maleated derivative of polyethylene, a polyamide (PA), a polyether block amide (PEBA), a nylon, a polyurethane (PU), an ethylene vinyl acetate (EVA), and copolymers or derivatives thereof.

Embodiment 10: The coated tubing of any of Embodiments 8 or 9, wherein the expansive component comprises EXPANCEL®.

Embodiment 11: The coated tubing of any of Embodiments 1-10, selected from the following: a coated tubing with an inner diameter of 0.015, wherein the expandable tie layer coating can expand about 10% or more in outer diameter when subjected to heat; a coated tubing with an inner diameter of 0.071, wherein the expandable tie layer coating can expand about 8% or more in outer diameter when subjected to heat; a coated tubing with an inner diameter of 0.138, wherein the expandable tie layer coating can expand about 12% or more in outer diameter when subjected to heat; and a coated tubing with an inner diameter of 0.338, wherein the expandable tie layer coating can expand about 5% or more in outer diameter when subjected to heat.

Embodiment 12: A tubing comprising an expanded tie layer thereon, comprising the coated tubing of any of Embodiments 1-11, wherein the expandable tie layer has been subjected to expansion to give the expanded tie layer.

Embodiment 13: A construction comprising the tubing of Embodiment 12 and an adjacent layer or component overlying and in intimate contact with at least a portion of the expanded tie layer, wherein the expanded tie layer is chemically bonded to the adjacent layer or component.

Embodiment 14: The construction of Embodiment 13, wherein the adjacent component is a metallic or polymeric tube (e.g., a laser-cut or straight or plain hypotube).

Embodiment 15: The construction of Embodiment 13, wherein the adjacent component comprises a braided structure, a coiled structure, or a fiber-reinforcing component.

Embodiment 16: The construction of any of Embodiments 13-15, where the expanded tie layer coating is chemically bonded around 360 degrees of the tubing to the adjacent layer or component.

Embodiment 17: The construction of any of Embodiments 13-16, wherein the expanded tie layer coating extends into and/or through the adjacent layer or component.

Embodiment 18: The construction of Embodiment 17, wherein the expanded tie layer coating extends through the adjacent layer or component, optionally so as to form an outer jacket of the construction.

Embodiment 19: A catheter comprising the tubing of Embodiment 12 or the construction of any of Embodiments 13-18, configured such that the catheter has a catheter inner surface corresponding to the inner surface of the tubing.

Embodiment 20: A modified stent comprising the tubing of Embodiment 12 or the construction of any of Embodiments 13-18.

Embodiment 21: A modified stent, comprising: a stent, comprising an inner surface and an outer surface, and a modified membrane, the membrane comprising an expanded tie layer coating on a surface thereof, prepared from an expandable tie layer coating that has been subjected to expansion, wherein the expanded tie layer is in contact with the inner surface of the stent such that the stent is encapsulated by the modified membrane.

Embodiment 22: The modified stent of Embodiment 21, wherein the expanded tie layer coating extends through the stent at least to the outer surface.

Embodiment 23: A modified stent, comprising: a stent, comprising an inner surface and an outer surface, and a modified membrane, the membrane comprising an expanded tie layer coating on a surface thereof, prepared from an expandable tie layer coating that has been subjected to expansion, wherein the expanded tie layer coating is in contact with the outer surface of the stent.

Embodiment 24: The modified stent of any of Embodiments 20-23, wherein the membrane comprises a material selected from the group consisting of PTFE, ePTFE, PE, ePE (expanded polyethylene), and composites thereof.

Embodiment 25: The modified stent of any of Embodiments 20-24, wherein the stent comprises stainless steel or nitinol.

Embodiment 26: The modified stent of any of Embodiments 20-25, wherein the expanded tie layer coating comprises a bonding agent and an expansive component.

Embodiment 27: The modified stent of Embodiment 26, wherein the bonding agent comprises a polymer selected from a maleated derivative of polyethylene, a polyamide (PA), a polyether block amide (PEBA), a nylon, a polyurethane (PU), an ethylene vinyl acetate (EVA), and copolymers or derivatives thereof.

Embodiment 28: The modified stent of Embodiment 26 or 27, wherein the expansive component comprises EXPANCEL®.

Embodiment 29: A covered stent, comprising the modified stent of any of Embodiments 20-28, further comprising an outer covering, wherein the modified stent is adhered to the outer covering via the expanded tie layer.

It will be apparent to those skilled in the art that other embodiments of the invention are possible and that the examples presented here are not intended to be exhaustive. These and other features, aspects, and advantages of the present disclosure will be apparent from a reading of the following detailed description together with the accompanying drawings, which are briefly described below. The present disclosure includes any combination of two, three, four, or more features or elements set forth in this disclosure or recited in any one or more of the claims, regardless of whether such features or elements are expressly combined or otherwise recited in a specific embodiment description or claim herein. This disclosure is intended to be read holistically such that any separable features or elements of the disclosure, in any of its aspects and embodiments, should be viewed as intended to be combinable, unless the context of the disclosure clearly dictates otherwise.

### DETAILED DESCRIPTION

The present invention will now be described more fully hereinafter with reference to certain examples, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The present disclosure generally provides a modified catheter liner, which comprises an expandable tie layer on at least a portion of a surface (e.g., an outer surface) of a catheter liner or other tubing (also referred to herein as a “heat grow tie layer”). The expandable/heat grow tie layer comprises a bonding agent (e.g., comprising a polymer optionally modified, e.g., to provide a maleic anhydride-grafted (“maleated”) polymer) and an “expansive component,” such that the tie layer is designed to expand or “heat grow” up to and, in some cases, into or even through, adjacent components/layers. Non-limiting adjacent component layers include, e.g., an adjacent hypotube wall (where, in some embodiments, the expandable tie layer can expand into at least a portion of the kerfs therein) and/or an adjacent polymeric layer, with certain example adjacent components/layers including, but not limited to, outer jackets, braid layers, fiber layers, and/or coil layers.

By expanding or “heat growing” up to and/or into and/or through adjacent components/layers, the modified catheter liners provided herein can provide constructions exhibiting excellent adhesion of the liner to adjacent components/layers as referenced above, e.g., a hypotube and/or an outer jacket (via the expanded tie layer). The bonding agent within the expandable tie layer can provide a chemical bond to both metallic and polymeric substrates (e.g., adjacent components/layers). The expansive component is meant to provide intimate contact with the adjacent components/layers to enhance the adhesion. In some embodiments, the bond and/or contact between the expandable tie layer (after expansion) and the adjacent component(s)/layer(s) is present around the full 360 degrees of the circumference of the modified catheter liner, i.e., a “continuous bond” is present around the outer circumference of the modified liner and the adjacent component(s)/layer(s). In some embodiments, the bond and/or contact may be present around less than the full 360 degree circumference.

The composition, size, and features of the underlying catheter liner can vary. In some embodiments, the catheter liner that is modified according to the present disclosure comprises polytetrafluoroethylene (PTFE) and/or poly(ethylene) (PE). In some embodiments, the catheter liner comprises a PFAS-free material. In some embodiments, the catheter liner that is modified according to the present disclosure is a commercially available catheter liner. In some embodiments, the principles outlined herein allow for the use of catheter liners comprising materials that are often considered too sticky or tacky to be used within conventional constructions, e.g., including, but not limited to, polyether block amides (PEBAs), including softer durometer PEBA materials (e.g., Pebax® 25D) and polyurethanes (PUs), including softer durometer thermoplastic PUs such as Tecoflex® 80A, Tecoflex® 93A, Pellethane® 2363, and Neusoft. The catheter liners can be of various types and intended for various applications (e.g., including, but not limited to, neurovascular catheters). In some embodiments, the catheter liner is not chemically or physically modified other than by application of the expandable tie layer coating described herein; for example, in some embodiments, no etching is required to provide sufficient adhesion of the expandable tie layer coating thereto. In some embodiments, the catheter liner can be modified, e.g., via chemical etching to enhance adhesion/bonding of the expandable tie layer coating thereto.

The bonding agent is not particularly limited but is typically selected so as to provide some degree of bonding to an adjacent component/layer as referenced herein. In some embodiments, the bonding agent can be selected so as to ensure desired flexibility and/or bond strength in the final product. In some embodiments, the bonding agent can comprise, a polymer such as a polyethylene (PE), a polyamide (PA), such as a polyether block amide (PEBA), a polyurethane (PU), nylon, ethyl vinyl acetate (EVA), or a co-polymer or derivative thereof. EVA, where used, can include, for example, RESILOK®.

In some embodiments, the bonding agent comprises a derivative of one or more such polymers comprising suitable modifications to enhance bonding with the adjacent component(s)/layer(s), e.g., polymers comprising coupling agents grafted thereon. In certain embodiments, the bonding agent comprises a maleated derivative of one or more of the polymers referenced herein, for example, a maleated PE (e.g., maleic anhydride-grafted high-density polyethylene (MA-g-HDPE) or maleic anhydride-grafted low-density polyethylene (MA-g-LDPE). In some embodiments, maleated derivatives can advantageously enhance bonding with adjacent components/layers, including, in some embodiments, metals, e.g., so as to provide a chemical bond between the tie layer coating and a metal component without significant delamination. Various grades of such polymers can be used in embodiments of the disclosure (including polymers of various molecular weights). Maleated derivatives provided herein can comprise any amount of maleic anhydride grafted onto the polymer backbone.

The expansive component can be any material that can expand and/or foam upon the addition of heat. In some embodiments, the expansive component comprises acrylonitrile and, in some embodiments, is EXPANCEL® (available from Nouryon in the Netherlands), which is a lightweight filler/blower comprising thermoplastic microspheres encapsulating a gas. When heat is added, the gas within the microspheres of EXPANCEL® expands and the surrounding shell softens, providing an increase in volume. EXPANCEL® in unexpanded form can advantageously be used to form the catheter constructions described herein, such that the final catheter construction comprises EXPANCEL® in expanded form. Although the disclosure exemplifies this material as the expansive component, the disclosure is not intended to be read as being limited thereto; other materials that can expand upon the introduction of heat (e.g., foaming agents) can be used instead of or in addition to the EXPANCEL®.

The expandable tie layer on at least a surface of the catheter liner, comprising a bonding agent and expansive component can, in some embodiments, comprise a homogenous mixture of the bonding agent and the expansive component. The amounts and ratios of the two components are not particularly limited so long as generally, sufficient bonding agent is present so as to provide some amount of bonding to an adjacent surface after expansion of the tie layer and so long as, generally, sufficient expansive component is present to expand the tie layer to allow for expansion/foaming, e.g., to provide intimate contact with one or more adjacent layers of the catheter construction. The expandable tie layer, in some embodiments, consists essentially of the bonding agent and the expandable component. The expandable tie layer, in some embodiments, comprises the bonding agent and the expandable component, and may optionally comprise one or more additional components.

In some embodiments, the disclosure provides one or more constructions comprising the modified catheter liner described herein. For example, in some embodiments, a catheter construction or catheter comprising one or more other layers or components along with the modified catheter liner is provided. In some embodiments, the catheter construction or catheter comprises a braided or coiled wire reinforcing layer on an outer surface of the modified catheter liner as described herein. In some embodiments, the catheter construction comprises a metallic or polymeric structure overlying and in intimate contact with at least a portion of the modified catheter liner (i.e., with the expanded tie layer coating), wherein the expanded tie layer coating is chemically bonded to the metallic or polymeric structure. The metallic or polymeric structure can be, e.g., a metallic or polymeric tube, such as a straight or laser-cut tube or can be a coiled structure. In some embodiments, a catheter construction or catheter is provided that comprises a catheter jacket overlying one or more of these components. In some embodiments, the inner surface (ID) of the modified catheter liner is the catheter construction inner surface and/or the catheter inner surface.

Advantageously, in some embodiments, the flexibility and/or lubricity of the modified catheter liner provided herein is not significantly impacted by the modification described (i.e., by the inclusion of an expandable tie layer thereon, in unexpanded or expanded form).

In one non-limiting embodiment, a modified polymeric liner (e.g., a PTFE liner), comprising the bonding agent and EXPANCEL® as described herein is provided for use in a neurovascular catheter. A modified catheter liner as provided herein can be prepared as follows (which is understood to be one, non-limiting example of preparing the types of modified liners provided herein). The bonding agent is dissolved in a suitable solvent to give a polymer solution, and a dispersion of the expansive component (in a solvent, which can be the same as or different than the solvent of the polymer solution) is added to the polymer solution. The solvent can, in some embodiments, be selected from xylene, decalin, methyl n-amyl ketone, n-butyl propionate, isobutyl isobutyrate, and combinations thereof. One or both of the solution and dispersion can optionally be warmed/heated before combining them. The catheter liner is then dipped in the resulting mixture, and the solvent is flashed off, e.g., at 200° F. The temperature is then raised to 350° F. for two minutes, and then it is possible to remove the mandrel, resulting in a chemically bonded modified catheter liner (which, in some embodiments, can be further processed/modified to provide it as a component of a construction, e.g., catheter as provided herein).

In some embodiments the “heat grow tie layer” is created using a dispersion of the expansive component and the bonding agent as a coating for a liner that when heat is applied, will foam/expand. The disclosure includes both unexpanded expandable tie layer coatings (comprising an unfoamed material) and expanded/foamed tie layer coatings (comprising a foamed material with increased size/volume outwards from the outer diameter of the coated liner relative to the unfoamed material, generally referred to herein as “expanded tie layers”). As provided herein, the foaming/expansion can, in some embodiments, be sufficient to allow the expandable tie layer to come into contact with (or into or even through) at least a portion of an adjacent layer/component when in expanded form (i.e., in the form of an expanded tie layer). For example, in some embodiments, it may be sufficient to allow the expandable tie layer to foam/expand at least up to the ID of a surrounding hypotube, where present. In some embodiments, it may be sufficient to allow the expandable tie layer to foam/expand into at least a portion of the kerfs present in a surrounding hypotube, where present.

In some embodiments, a catheter liner (e.g. a PTFE liner) can be coated during the extrusion process. The modified catheter liner can then be loaded onto a mandrel and stretched down to keep the ID of the catheter (upon completion) tight to its specific size. This mandrel and modified catheter liner can be loaded into a hypotube, e.g., a laser-cut or straight or plain hypotube, which can be metallic or polymeric. After applying heat to the assembly, the expandable tie layer coating on the liner will expand/foam at least up to the ID of the hypotube and create a chemical bond therewith.

The degree of foaming/expansion can depend, in part, on the composition of the expandable tie layer (e.g., the content of expansive component) and the conditions under which it is expanded. As referenced herein, in some embodiments, the degree of foaming/expansion is sufficient such that the expanded tie layer fills any gap originally present between the liner and one or more adjacent layers/components applied thereto and can result in sufficient contact to ensure some degree of bonding between the tie layer and the adjacent layers/components. The desired degree of foaming/expansion will thus depend on the physical parameters of the liner and the adjacent layers/components. In some embodiments, the degree of foaming/expansion is sufficient for the expandable layer to foam/expand at least about 0.001″, at least about 0.002″, at least about 0.005″, at least about 0.01″, at least about 0.02″, at least about 0.03″, at least about 0.04″, at least about 0.05″, at least about 0.06″, at least about 0.07″, or at least about 0.08″ outwards from the outer diameter of the coated liner. In certain embodiments, the coated liner in expanded form can cover a gap from the outer diameter of the liner to a next adjacent layer/component of about 0.002″ to about 0.200,″ about 0.002″ to about 0.100,″ about 0.003″ to about 0.05″, about 0.002″ to about 0.01″, about 0.002″ to about 0.005″, about 0.003″ to about 0.005″, about 0.005″ to about 0.100″, about 0.005″ to about 0.01″, about 0.01″ to about 0.100″, about 0.01″ to about 0.05″, about 0.01″ to about 0.03.″ In some embodiments, the coated liner in expanded form can cover a gap of 0.002″ to about 0.1″ from the OD of the liner to an inner diameter of a surrounding component of a catheter, e.g., a hypotube.

In some embodiments, when the inner diameter (ID) of the modified liner with the expandable tie layer coating is between 0.015-0.5 inches, the expansive component can provide an expansion (e.g., a free expansion, without any intervening layers) of at least about 5%, at least about 10%, at least about 15%, or at least about 20% (e.g., about 5% to about 65%, about 5% to about 60%, about 5% to about 50%, about 5% to about 40%, about 5% to about 30%, about 5% to about 20%, or about 5% to about 15% in various embodiments or about 10% to about 65%, about 10% to about 60%, about 10% to about 50%, about 10% to about 40%, about 10% to about 30%, or about 10% to about 20%) in various embodiments. The expansion referenced herein is a percent expansion of the outer diameter of the liner+expandable (but unexpanded) tie layer coating as compared with the outer diameter of the liner+expanded tie layer coating.

The expansion provided by a given expandable tie layer can depend, for example, on the ID of the liner, with certain modified liners exhibiting greater expansion when a roughly equivalent content of expandable tie layer material (comprising a roughly equivalent content of expansive component) is applied thereto. For example, for an expandable tie layer coating on a catheter liner with an ID of 0.015 inches, the expansive component may provide an expansion of greater than 10%, e.g., about 10% to about 65%. For an expandable tie layer coating on a catheter liner with an ID of 0.071 inches, the expansive component may provide an expansion of greater than 8%, e.g., about 8% to about 60%. For an expandable tie layer coating on a catheter liner with an ID of 0.138 inches, the expansive component may provide an expansion of greater than 10%, e.g., about 10% to about 25%. For an expandable tie layer coating on a catheter liner with an ID of 0.338 inches, the expansive component may provide an expansion of greater than 5%, e.g., about 5% to about 15%. It is to be noted that the disclosure is not limited to modified catheter liners only with the referenced ID values; these are simply examples of possible expansions associated with certain specific expandable tie layers on certain, non-limiting catheter liners.

In some embodiments, the percent expansion increases with an increased amount of expansive component applied to the surface of the liner (e.g., by contacting the liner with a solution/dispersion of the expansive component and the bonding agent comprising a higher concentration of the expansive component). In some embodiments, the percent expansion can approximately double when the catheter liner is contacted with a solution/dispersion containing double the amount of expansive component by weight, e.g., from 0.5% to 1% by weight in solution/dispersion). Such an observation may, in some embodiments, be particularly striking when the ID of the catheter liner is between about 0.05 inches and 0.4 inches. In some embodiments, the percent expansion can more than double when a liner is contacted with a solution/dispersion containing double the amount of expansive component by weight, e.g., from 1% to 1.5% by weight in solution/dispersion. Such an observation may, in some embodiments, be particularly striking when the ID of the catheter liner is about 0.015 inches.

In some embodiments, the expandable tie layer can be foamed/expanded so as to act as an outer jacket for a catheter construction. For example, in one embodiment, the expandable tie layer is formed and expanded up to and through a braid or coil, such that it forms an outer jacket of the construction. In some embodiments, such a construction may allow for replacement of a dip coated braided or coiled shaft.

In some embodiments, a membrane comprising the expandable tie layer described herein on an outer surface thereof can be expanded to (and optionally, into and/or through) the inner surface of a stent in order to encapsulate the stent and provide adhesion to an outer covering. In other embodiments, a membrane comprising the expandable layer on an inner surface can be used as an outer covering of an encapsulated stent, e.g., via wrapping the stent with the modified membrane. The coverings can comprise membranes made out of expanded PTFE and expanded polyethylene as well as composite membranes. The stent can comprise a number of materials such as, for example, stainless steel and nitinol.

In some embodiments, the disclosure provides the formulation of the bonding agent/expansive component dispersion coating of a polymeric (e.g., PTFE or PE) liner. The expansive component in its powder form is dispersed in a solvent, e.g., including, but not limited to, xylene, decalin, methyl n-amyl ketone, n-butyl propionate, or isobutyl isobutyrate, and heated, e.g., to around 90° C. A bonding agent as described above is added, e.g., incrementally. Once all bonding agent is added, the temperature of the dispersion is maintained at an elevated temperature, e.g., at 90° C. for a period of time, e.g., 90 minutes.

In some embodiments, the methods and materials provided herein can provide a catheter assembly that exhibits suitable delamination properties between the expanded tie layer (i.e., after heating) and an adjacent catheter component (e.g., a hypotube) to render it useful for the desired application. In some embodiments, the delamination is comparable to or better than the delamination exhibited by conventional tie layers and associated hypotubes.

### EXPERIMENTALS

EXPANCEL® microspheres were obtained from Nouryon. These were used as the expansive component in the heat grow tie layer solution. The expansive component in its powder microsphere form was dispersed in xylene and heated to around 90° C. A RESILOK®EVA tie layer bonding agent material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ liner tube with a specified ID was hand-pulled through a pot with skimming dies in order to coat the tube with the heat grow tie layer solution. The tube was then allowed to dry for 24 h before the dimensions of the inner diameter (ID) and the outer diameter (OD) of the tie-layer coated and uncoated liner tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated liner end and coated, expanded tie layer plus liner end were measured using a Keyence VHX-5000 microscope at 100× and 500× magnification. The percent expansion of the liner plus expanded tie layer was then calculated by comparing the OD of the coated, unexpanded tie layer plus liner tube end to the OD of the coated, expanded tie layer plus liner tube end. This example was repeated three times. Liner plus tie layer expansion results are provided in Table 1.

It was observed for these examples that optimal expansion occurred at 1.0% loadings of the expansive component in the coating stage and that for IDs greater than 0.015 inches and less than 0.500 inches, the expansion of the tie layer approximately doubled when the EXPANCEL® loading increased from 0.5% to 1.0%. The tube of the largest ID tested (0.500 inches) expanded the least and indicated that larger tube IDs may require an EXPANCEL® loading of about 1.5% to achieve expansion comparable to that seen for the catheters tested with lower IDs.

In order to determine the force required to cause delamination of the liner plus tie layer expanded onto a hypotube, a PTFE Sub-Lite-Wall™ liner tube (0.0175″ ID+/−0.002″ with a wall thickness of 0.0015″+/−0.0005) was coated with 1.0% heat grow tie layer solution and allowed to dry. The modified liner (comprising the liner plus tie layer thereon) was then inserted into a hypotube (allowing for a 3 inch overhang where the liner was exposed, i.e., where the hypotube was not around the liner plus tie layer), and the assembly was heated in an oven at 325° F. for 10 minutes to enable the tie layer to expand into the kerfs of the hypotube. The 3 inch overhang of liner plus tie layer was then placed in the top clamp of an Instron (Model 3340 series, single column table frames) equipped with a 100N load cell, and the tube end where the liner plus expanded tie layer plus hypotube was placed in the bottom clamp. The initial gauge length was 6 inches, and the Instron pulled the tube sample at a rate of 1 inch per minute until failure occurred. This test was repeated two times, and compared to the average delamination force (of 2 replicates) of a commercially available PEBAX tie layer+heat grow mandrel used with the same liner and hypotube construction as described above and compared to the average delamination force (of 2 replicates) of a commercially available PEBAX® tie layer+air assist used with the same liner and hypotube construction as described above. In all cases, the liners failed (tore in the overhang region) before any signs of delamination were observed. Therefore, the delamination force for the Heat Grow Tie Layer is considered to be comparable to that of a commercially available PEBAX® tie layer.

Overall, results suggest that a catheter liner plus an expandable tie layer that is capable of expanding e.g., into the kerfs of a hypotube after being inserted into a hypotube and heated provides more options for catheter constructions as less clearance is needed between the OD of the modified catheter liner and the ID of the hypotube. Also, since more clearance flexibility is possible, it is easier to use tackier materials for liners, thus expanding the possible materials that can be used for liners beyond fluoropolymers, e.g., to PU and other PEBA materials as referenced herein above.

Aspects of the present disclosure are more fully illustrated by the following examples, which are set forth to illustrate certain aspects of the present invention and are not to be construed as limiting thereof.

### Comparative Example 1

A heat grow tie layer solution was prepared with 0.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.500″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Comparative Example 2

A heat grow tie layer solution was prepared with 1.0% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.500″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Comparative Example 3

A heat grow tie layer solution was prepared with 1.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.500″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 1

A heat grow tie layer solution was prepared with 0.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.015″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 2

A heat grow tie layer solution was prepared with 1.0% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.015″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 3

A heat grow tie layer solution was prepared with 1.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.015″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 4

A heat grow tie layer solution was prepared with 0.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.071″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 5

A heat grow tie layer solution was prepared with 1.0% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.071″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 6

A heat grow tie layer solution was prepared with 1.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.071″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 7

A heat grow tie layer solution was prepared with 0.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.138″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 8

A heat grow tie layer solution was prepared with 1.0% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.138″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 9

A heat grow tie layer solution was prepared with 1.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.138″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 10

A heat grow tie layer solution was prepared with 0.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.338″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 11

A heat grow tie layer solution was prepared with 1.0% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.338″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

### Example 12

A heat grow tie layer solution was prepared with 1.5% EXPANCEL® dispersed in xylene and heated to 90° C. A RESILOK®EVA tie layer material grafted with maleic anhydride (MA-g-EVA) was then added incrementally until the dispersion contained a vinyl acetate content of about 15%. Once all EVA was added, the temperature of the dispersion was maintained at 90° C. for 90 minutes. The bottom half of a PTFE Sub-Lite-Wall™ tube with a 0.338″ ID was hand-pulled through a pot with skimming dies in order to coat the tube with the heat grow tie layer solution. The tube was then allowed to dry for 24 h before the dimensions of the inner diameter (ID) and the outer diameter (OD) of the coated and uncoated tube ends were measured using a Keyence VHX-5000 microscope at 100× and 500× magnification. The tube was then heated in an oven at 325° F. for 10 minutes to enable expansion. The tube was allowed to cool fully before the dimensions of the inner diameter (ID) and the outer diameter (OD) of the uncoated end and coated, expanded end were measured using a Keyence VHX-5000 microscope at 100× and 500× magnification. The percent expansion of the liner plus expanded tie layer was then calculated by comparing the OD of the coated, unexpanded tube end to the OD of the coated, expanded tube end. This example was repeated three times. Liner plus tie layer expansion results are provided in Table 1.

TABLE 1
Outer Diameter (OD) Expansion of the Liner + Tie Layer

| | General ID (in) | % EXPANCEL ® | % Expansion |
|---|---|---|---|
| Example | Tested | loading | of OD |
| 1 | 0.015 | 0.5% | 62% |
| 2 | 0.015 | 1.0% | 12% |
| 3 | 0.015 | 1.5% | 30% |
| 4 | 0.071 | 0.5% | 8% |
| 5 | 0.071 | 1.0% | 17% |
| 6 | 0.071 | 1.5% | 56% |
| 7 | 0.138 | 0.5% | 12% |
| 8 | 0.138 | 1.0% | 21% |
| 9 | 0.138 | 1.5% | 22% |
| 10 | 0.338 | 0.5% | 5% |
| 11 | 0.338 | 1.0% | 11% |
| 12 | 0.338 | 1.5% | 9% |
| Comp 1 | 0.500 | 0.5% | 2% |
| Comp 2 | 0.500 | 1.0% | 4% |
| Comp 3 | 0.500 | 1.5% | 11% |

Many modifications and other embodiments of the invention will come to mind to one skilled in the art to which this invention pertains having the benefit of the teachings presented in the foregoing description. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

What is claimed is:

1. A coated tubing comprising:
a polymeric tubing, the polymeric tubing having an inner surface and an outer surface and a wall thickness; and
an expandable tie layer coating on at least a portion of the outer surface that can expand about 5% or more in outer diameter when subjected to heat.

2. The coated tubing of claim 1, wherein the expandable tie layer can expand about 10% or more in outer diameter when subjected to heat.

3. The coated tubing of claim 1, wherein the expandable tie layer can expand about 15% or more in outer diameter when subjected to heat.

4. The coated tubing of claim 1, wherein the expandable tie layer can expand up to about 65% in outer diameter when subjected to heat.

5. The coated tubing of claim 1, wherein the polymeric tubing comprises polytetrafluoroethylene (PTFE) or polyethylene (PE).

6. The coated tubing of claim 1, wherein the polymeric tubing comprises polyurethane (PU) or polyether block amide (PEBA).

7. The coated tubing of claim 1, wherein the expandable tie layer coating is on substantially all of the outer surface.

8. The coated tubing of claim 1, wherein the expandable tie layer coating comprises a bonding agent and an expansive component.

9. The coated tubing of claim 8, wherein the bonding agent comprises a polymer selected from a maleated derivative of polyethylene, a polyamide (PA), a polyether block amide (PEBA), a nylon, a polyurethane (PU), an ethylene vinyl acetate (EVA), and copolymers or derivatives thereof.

10. The coated tubing of claim 8, wherein the expansive component comprises EXPANCEL®.

11. The coated tubing of claim 1, selected from the following:
a coated tubing with an inner diameter of 0.015, wherein the expandable tie layer coating can expand about 10% or more in outer diameter when subjected to heat;
a coated tubing with an inner diameter of 0.071, wherein the expandable tie layer coating can expand about 8% or more in outer diameter when subjected to heat;
a coated tubing with an inner diameter of 0.138, wherein the expandable tie layer coating can expand about 12% or more in outer diameter when subjected to heat; and
a coated tubing with an inner diameter of 0.338, wherein the expandable tie layer coating can expand about 5% or more in outer diameter when subjected to heat.

12. A tubing comprising an expanded tie layer thereon, comprising the coated tubing of claim 1, wherein the expandable tie layer has been subjected to expansion to give the expanded tie layer.

13. A construction comprising the tubing of claim 12 and an adjacent layer or component overlying and in intimate contact with at least a portion of the expanded tie layer, wherein the expanded tie layer is chemically bonded to the adjacent layer or component.

14. The construction of claim 13, wherein the adjacent component is a metallic or polymeric tube (e.g., a laser-cut or straight or plain hypotube) or wherein the adjacent component comprises a braided structure, a coiled structure, or a fiber-reinforcing component.

15. The construction of claim 13, wherein the expanded tie layer coating extends into and/or through the adjacent layer or component, optionally so as to form an outer jacket of the construction.

16. A catheter comprising the construction of claim 13, configured such that the catheter has a catheter inner surface corresponding to the inner surface of the polymeric tubing.

17. A modified stent, comprising:
a stent, comprising an inner surface and an outer surface, and
a modified membrane, the membrane comprising an expanded tie layer coating on a surface thereof, prepared from an expandable tie layer coating that has been subjected to expansion,
wherein the expanded tie layer is in contact with:
the inner surface of the stent such that the stent is encapsulated by the modified membrane; and/or
the outer surface of the stent.

18. The modified stent claim 17, wherein the expanded tie layer coating comprises a bonding agent and an expansive component.

19. The modified stent of claim 18, wherein the bonding agent comprises a polymer selected from a maleated derivative of polyethylene, a polyamide (PA), a polyether block amide (PEBA), a nylon, a polyurethane (PU), an ethylene vinyl acetate (EVA), and copolymers or derivatives thereof.

20. The modified stent of claim 18, wherein the expansive component comprises EXPANCEL®.
